(19) United States
(12) Patent Application Publication
(10) Pub. No.: US 2010/0145155 A1
(43) Pub. Date: Jun. 10, 2010

(54) ABDOMINAL RETRACTION FOR MEDICAL/SURGICAL PROCEDURES

(75) Inventor: Paul Sorajja, Rochester, MN (US)

Correspondence Address: SHUMAKER & SIEFFERT, P. A., 1625 RADIO DRIVE, SUITE 300, WOODBURY, MN, 55125, US

(73) Assignee: Mayo Foundation for Medical Education and Research

(21) Appl. No.: 12/629,368

(22) Filed: Dec. 2, 2009

Related U.S. Application Data
(60) Provisional application No. 61/120,699, filed on Dec. 8, 2008.
(60) Provisional application No. 61/163,662, filed on Mar. 26, 2009.

(51) Int. Cl. A61B 17/00

(52) U.S. Cl. 600/235

(57) ABSTRACT

A device retracts abdominal and/or inguinal pannus away from the inguinal region of a patient. The device includes a garment generally in the shape of a girdle or athletic shorts. The device includes a front side and a back side, and a lower portion including a pair of legs that extend from the upper portion, the garment further including first and second apertures positioned on the front side of the garment such that the first and second apertures expose the right and left inguinal regions of a patient, respectively, when the patient wears the garment. The device may be useful for endovascular procedures requiring access to the femoral arteries and femoral veins of a patient, or during other procedures that require access to the inguinal region of a patient.

This application claims the benefit of U.S. Provisional Application No. 61/120,699 entitled “ABDOMINAL RETRACTION FOR MEDICAL/SURGICAL PROCEDURES,” and filed on Dec. 8, 2008, and U.S. Provisional Application No. 61/163,662 entitled “ABDOMINAL RETRACTION FOR MEDICAL/SURGICAL PROCEDURES,” and filed on Mar. 26, 2009. The entire content of each of these provisional applications is incorporated herein by reference.

### TECHNICAL FIELD

The invention relates to abdominal retraction devices worn by a patient during medical and/or surgical procedures, such as endovascular procedures requiring access to the femoral arteries and femoral veins of a patient, or during other procedures that require access to the inguinal region of a patient.

### BACKGROUND

Cardiac, radiologic, and other endovascular procedures routinely require access to the femoral arteries and femoral veins. However, proper draping and exposure of the inguinal region while maintaining patient modesty and procedural sterility can be challenging in many patients, particularly in obese patients with large body habitus. Many such patients have developed what is known as an abdominal pannus, an overhanging flap of abdominal tissue consisting of skin, fat, and sometimes contents of the internal abdomen. Inguinal and thigh pannus folds may also be present. In obese patients, pannus folds can partially or completely overlap the inguinal region. These extraneous pannus folds may make access to the femoral artery and/or groin area difficult for the surgeon or medical practitioner, interfere with proper sterilization of the area, lead to mechanical vascular complications by causing the operator to falsely interpret landmarks for vascular access, and/or cause patient discomfort because of the need to retract the pannus with tape or other means that are not purpose-dedicated.

### SUMMARY

In general, the invention provides a device that retracts abdominal and/or inguinal pannus away from the inguinal region of a patient. The device may be useful for endovascular procedures requiring access to the femoral arteries and femoral veins of a patient, or during other procedures that require access to the inguinal region of a patient.

In one embodiment, the invention is directed to apparatus comprising a garment including an upper portion that defines a waist opening and having a front side and a back side, and a lower portion including a pair of legs that extend from the upper portion, the garment further including first and second apertures positioned on the front side of the garment such that the first and second apertures expose the right and left inguinal regions of a patient, respectively, when the patient wears the garment. The garment may further include a first upper fastening mechanism positioned on the right front side of the upper portion and a second upper fastening mechanism positioned on the left front side of the upper portion. The garment may further include a first lower fastening mechanism positioned on the right front side of the lower portion and a second lower fastening mechanism positioned on the left front side of the lower portion. The first upper fastening mechanism and the second upper fastening mechanism may each comprise at least one of a hook, a D-ring, a snap, a buckle, an anchor or a button. The first upper fastening mechanism and the second upper fastening mechanism may each comprise a fabric panel comprising a hook and loop fastener. The outside of the garment may further include adhesive regions surrounding first and second apertures, respectively. The inside of the garment may further include adhesive regions surrounding the first and second apertures, respectively. The apparatus may further include at least one retraction strap releasably connected to one of the fastening mechanisms.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a front view of an example abdominal retraction device as worn by a patient.

FIG. 2A is a side view of the example abdominal retraction device of FIG. 1 as worn by a patient before application of superior and/or inferior retraction of the abdominal tissue.

FIG. 2B is a side view of the example abdominal retraction device of FIG. 1 as worn by a patient after application of superior and/or inferior retraction of the abdominal tissue.

FIG. 3 is a front view of another example abdominal retraction device as worn by a patient.

FIG. 4 is a front view of another example abdominal retraction device as worn by a patient.

FIG. 5 is a front view of another example abdominal retraction device as worn by a patient.

FIG. 6 is a side view of the example abdominal retraction device of FIG. 5 as worn by a patient after application of retraction of the abdominal tissue.

FIG. 7 is a front view of another example abdominal retraction device as worn by a patient.

### DETAILED DESCRIPTION

The present invention provides a device that retracts abdominal and/or inguinal pannus away from the inguinal region. Cardiac, radiologic, and other endovascular procedures routinely require access to the femoral arteries and femoral veins. The femoral artery is a large artery that starts in the lower abdomen and goes down into the thigh. During such procedures, the femoral arteries or veins are commonly accessed at the inguinal (groin) region where the thigh meets the abdomen. However, proper draping and exposure of the inguinal region while maintaining patient modesty and procedural sterility can be challenging in many patients, particularly in obese patients with large body habitus who may have developed extraneous pannus folds of the abdomen or inguinal/thigh region. Indeed, abdominal or thigh/inguinal pannus can pose difficulties even in those patients who are only moderately overweight. Given the millions of endovascular procedures performed annually and the increasing prevalence of obesity in the general population, some way of providing unimpeded access to the inguinal region would be beneficial.

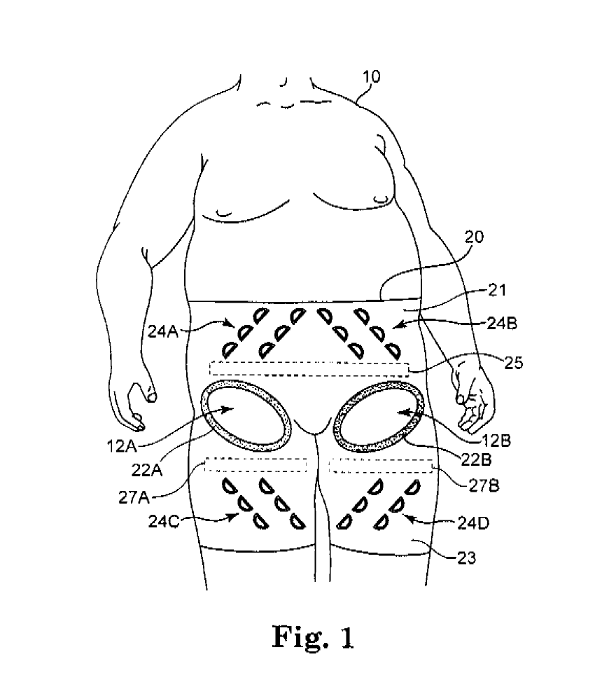

FIG. 1 is a front view of an example abdominal retraction device 20 as worn by a patient 10. Device 20 provides retraction of pannus away from the inguinal region, may facilitate maintenance of sterility during the vascular procedure and provides coverage and support of the patient's genital area. As shown in FIG. 1, device 20 may generally take the form of a close fitting garment similar to a girdle or pair of athletic shorts. Device 20 includes an upper portion 21 that defines a waist opening and a lower portion 23 having a pair of legs extending from upper portion 21 and that define corresponding leg openings.

Device 20 may be constructed of tightly woven, radiolucent material through which x-ray or other imaging remains possible. The material may be somewhat elastic to conform to the shape of the patient's body. The material need not be elastic, however. The strength and shape of the material lifts the pannus away from the inguinal region either superiorly, inferiorly, or both. In general, the upper and lower portions 21, 23 of abdominal retraction device 20, or other example abdominal retraction devices which shall become apparent to those of skill in the art upon reading and understanding this specification, may be constructed using a fabric having sufficient stiffness to adequately contain the abdominal and/or thigh pannus for purposes of ensuring access to the inguinal region of a patient. One such nonlimiting example is a fabric containing 30% spandex/70% lycra. However, it shall be understood that many other fabrics could also be used to construct an abdominal retraction device, and that the invention is not limited in this respect.

Device 20 may also be constructed to minimize patient irritation such as by using flat-locking stitching for any seams, ensuring that no seams are placed at key surgical areas, and/or placing labels on the outside of the garments, or other means of avoiding irritation. Device 20 may also be constructed of latex-free or other allergen free material.

When worn by a patient, upper portion 21 of device 20 (the abdominal portion generally covering the abdominal region of the patient) enables retraction of abdominal pannus in the superior direction (generally toward the head of the patient) while bottom portion 23 (the leg portion generally covering the thigh region of the patient) enables retraction of thigh and inguinal pannus in the inferior direction (generally toward the feet of the patient).

Further retraction of the pannus may be aided in a variety of ways. For example, device 20 may include variously located fastening mechanisms to which belts, tie downs or other straps may be attached, the use of bib or shoulder straps, the use of hook and loop fastener straps on upper 21 and lower portion 23, or any combination of these.

In the example shown in FIG. 1, device 20 includes multiple sets of fasteners 24A-24D to which belts, tie-downs or other straps can be attached to provide further retraction of abdominal and/or inguinal pannus. Fasteners 24A and 24B are positioned on a front side of upper portion 21 of device 20 to enable superior retraction of the abdominal pannus and fasteners 24C and 24D are positioned on the front side of lower portion 23 of device 20 to enable inferior retraction of the thigh/inguinal pannus.

Fasteners 24A-24D may include any of a variety of different types of fasteners. For example, fasteners 24A-24D may include hooks, D-rings, snaps, buckles, anchors, buttons, fixtures or any other mechanism that facilitates an attachment or connection. Fasteners 24A-24D may include all of the same type of fastener, or may include two or more different types of fasteners.

Fasteners 24A-24D may be fabricated from any suitable material, including metal, plastic, fabric, or other suitable material. In some examples, fasteners 24A-24D may be fabricated from a radiolucent material that allows passage of x-rays or other radiation for imaging or other purposes.

Each set of fasteners 24A-24D may include multiple fasteners, and may also include more than one row of fasteners to provide a flexible mechanism for applying a retraction force. In the example of FIG. 1, each set of fasteners 24A-24D includes two rows of three fasteners. However, it shall be understood that each set of fasteners may include one or more individual fasteners and/or one or more rows of fasteners, and that the invention is not limited in this respect.

Device 20 also includes first and second apertures 12A and 12B positioned on the front side of the garment such that the first and second apertures 12A and 12B expose the right and left inguinal regions of a patient, respectively. Apertures 12A and 12B thus provide access to the femoral arteries/veins of the patient 10 when the device 20 is worn by the patient. Adhesive regions 22A and 22B on the outside surface of device 20 may surround apertures 12A and 12B, respectively. Adhesive regions 22A and 22B include an adhesive material, such as glue, hook and loop fastener (e.g., Velcro), or other fastening mechanism, to which an overlying sterile drape may be attached to create a sterile barrier during the procedure. Adhesive regions 22A and 22B may continuously surround apertures 12A and 12B as shown in FIG. 1, or discrete segments of adhesive may be placed around each of apertures 12A and 12B. In addition or alternatively, adhesive may be placed on the inside surface of device 20 surrounding apertures 12A and 12B, such that the adhesive comes into contact with the patient's skin when device 20 is worn by the patient.

A mid-portion of the device provides patient modesty with complete genital coverage. Device 20 may also be constructed with a removable or retractable window over or around the genital region (not shown) to enable placement of urinary catheters during a procedure. This may be desirable to preserve patient modesty even for those patients who do not require pannus retraction during an endovascular procedure.

Device 20 may further include one or more reinforcing panels, such as reinforcing panels 25, 27A and/or 27B. The reinforcing panel(s) may be made from a fabric that is somewhat stiffer, heavier or less elastic that the fabric that makes up the rest of device 20. The purpose of reinforcing panel(s) is to help prevent excess stress around apertures 12A and 12B from application of retraction force that may result in a rip or tear in the fabric of device 20 around apertures 12A or 12B. Reinforcing panel 25, for example, may provide extra support to the fabric of device 20 when a retraction force is applied to fasteners 24A and/or 24B, and may serve to isolate this retraction force from the fabric on the superior side of apertures 12A and 12B. Similarly, reinforcing panels 27A and 27B, for example, may provide extra support to the fabric of device 20 when a retraction force is applied to fasteners 24C and/or 24D, and may serve to isolate this refraction force from the fabric on the inferior side of apertures 12A and 12B.

In use, device 20 may be put on by the patient in the preparatory area before the start of the procedure. During procedure preparation with the patient on a procedure table, such as a fluoroscopic table, device 20 may be further adjusted via belts or straps to enable retraction of the pannus and exposure of the right and/or left inguinal regions. The inguinal skin area is sterilized in the usual fashion. This may be followed by overlay of the routine sterile drape with attachment of the drape to adhesive regions 22A and 22B.

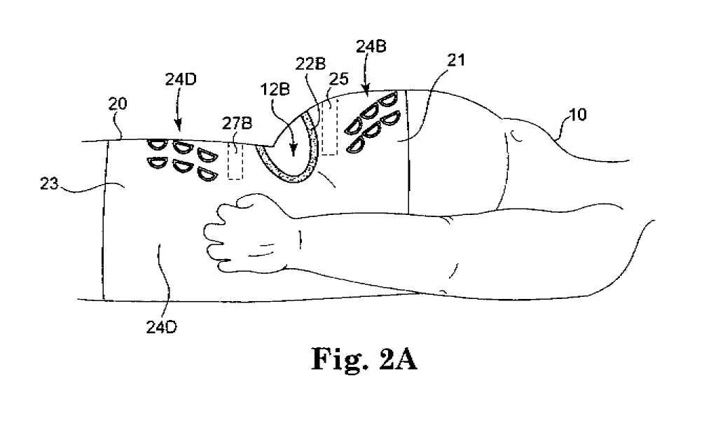

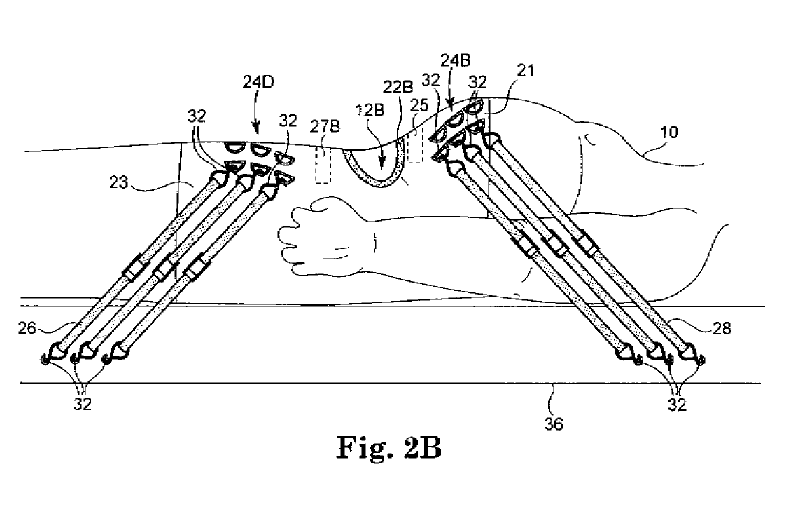

FIG. 2A is a side view of the example abdominal retraction device of FIG. 1 as worn by a patient before application of additional superior and/or inferior retraction of the abdominal tissue. In FIG. 2A, patient 10 wears device 20 and lies in a supine position upon a procedure table 36, such a fluoroscopic table. Depending upon the patient, wearing of the device 20 may by itself provide sufficient retraction of the abdominal and/or inguinal or thigh pannus to provide adequate access to the inguinal region. In other patients, further retraction may be required to adequately expose the inguinal region and provide sufficient access to the femoral arteries/veins.

FIG. 2B is a side view of an example abdominal retraction device 20 of FIGS. 1 and 2B as worn by a patient after application of additional superior and/or inferior retraction of the abdominal tissue. As in FIG. 2A, patient 10 wears device 20 and lies in a supine position upon procedure table 36. In this example, however, superior retraction of the abdominal tissue may be applied by one or more releasable belts or straps 28 (hereinafter “straps 28”). Straps 28 may include releasable connectors (such as hook-shaped connectors 32) that permit releasable attachment to fasteners 24 at one end and a fixed object (such as the procedure table 36) at the other end. To retract the abdominal pannus, a first end of a strap 28 is connected to fasteners 24A (not shown) and 24B using a releasable connector 32. Tension is applied to straps 28 in a generally superior direction to retract the abdominal pannus away from the inguinal region. A second end of straps 28 may then be secured to a fixed object via releasable connectors 32 to maintain tension in straps 28 and thus maintain the retraction of the abdominal pannus. In one example, such as that shown in FIG. 2B, the second end of straps 28 may be secured to procedure table 36 using cleats, rails or other attachment mechanisms on procedure table 36. In another example, the second end of straps 28 may be secured to some other fixed object in the procedure room, may be fastened to an appropriate location on device 20, or may be fastened or belted around the patient so as to maintain retraction of the abdominal pannus. In another example, straps 28 may loop through one or more hooks 24A or 24B and the loose ends may then connect to the fixed object via releasable connectors or via cleats, rails, or other attachment mechanism to maintain the retraction.

Similarly, inferior retraction of the inguinal/thigh tissue may be applied by one or more belts or straps 26. Releasable connectors 32 on the first end of straps 26 connect to fasteners 24C (not shown) and 24D. Tension is applied to straps 26 in a generally inferior direction to retract the inguinal/thigh pannus away from the inguinal region. Releasable connectors at the second end of straps 26 may then be secured to a fixed object to maintain tension in straps 26 and thus maintain the refraction of the inguinal/thigh pannus. In one example, the second end of straps 26 may be secured to the procedure table using cleats, rails or other attachment mechanisms on procedure table 36. In another example, the second end of straps 26 may be secured to some other fixed object in the procedure room, may be fastened to an appropriate location on device 20, or may be fastened or belted around the patient so as to maintain retraction of the inguinal/thigh pannus. In another example, straps 26 may loop through one or more hooks 24C or 24D and the loose ends may then be secured to the fixed object to maintain retraction.

Releasable straps 28 may be implemented with a wide variety of different mechanisms for applying and maintaining a retraction force. For example, belts, straps, tie-downs, ropes, bands, or other mechanism may be used. The straps may be made of elastic or non-elastic material, including nylon, polypropylene, or polyester webbing, fabric, etc. The straps may be adjustable or non-adjustable. For example, straps 28 may include an adjustment mechanism 34, such as adjustable, ratcheting, quick release, or retractable buckles, cams, cleats or other strap adjustment mechanism. The releasable connectors 32 may be implemented using S-hooks, J-hooks, snap hooks, clips, buckles or other mechanism for releasable attachment. Alternatively, rather than including releasable hardware at each end, straps 28 may be looped through fasteners 24 on device 20 and through fasteners on the fixed object and secured to itself using Velcro, hook and loop fasteners, snaps, buttons, clips etc. These examples are merely exemplary and it shall be understood that many other alternative types of releasable straps may be used, and that the invention is not limited in this respect.

Depending upon the procedure, either one or both inguinal regions 12A, 12B may need to be accessed by the physician. Thus, for some procedures, retraction may only be required of either the right or left abdominal pannus, the left or right inguinal/thigh pannus, some other combination or all of these may be required to provide the retraction required for that particular procedure. It shall be understood, therefore, that multiple configurations of device 20 may be constructed. In addition, it shall be understood that not all of fasteners 24A-24D need be used in every procedure, and that only those fasteners or other retraction mechanisms that provide the particular retraction required for that procedure need be used at any one time.

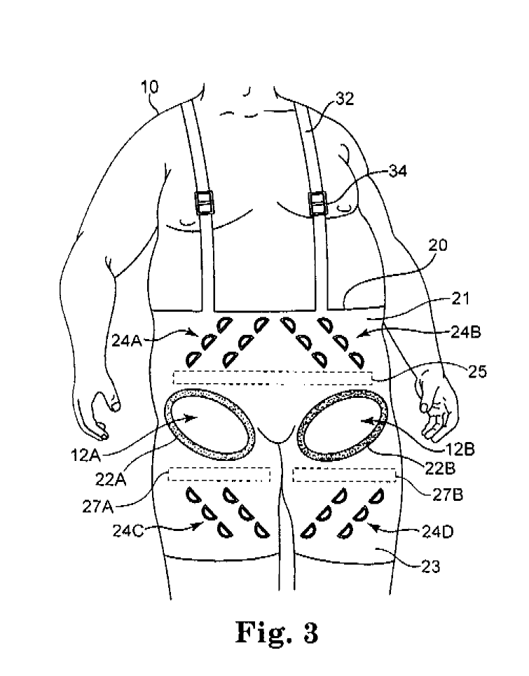

FIG. 3 is a front view of another example abdominal retraction device 30 as worn by a patient 10. In this example, device 30 includes shoulder straps 32 that are worn over the shoulders of patient 10. Shoulder straps 32 provide for additional superior retraction of the abdominal pannus and may be adjusted via an adjustment mechanism 34 such as adjustable, ratcheting, quick release, or retractable buckles, cams, cleats or other strap adjustment mechanism. The adjustment mechanism 34 may be placed anywhere along the length of shoulder straps 32.

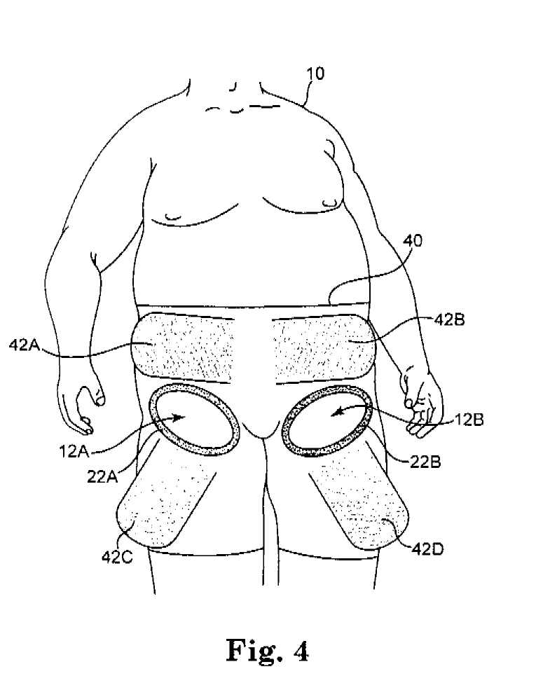

FIG. 4 is a front view of another example abdominal retraction device 40 as worn by a patient 10. In this example, device 40 includes multiple hook and loop fastener panels 42A, 42, 42C and 42D positioned to provide releasable retraction of the abdominal, inguinal and/or thigh pannus away from the inguinal region. For example, panels 42A and 42B may be positioned on the upper portion 21 of device 20, and fastener panels 42C and 42D may be positioned on the lower portion 23 of device 20. Panels 42A and 42B may be pulled generally upward and toward the back of the patient to retract the abdominal pannus away from the inguinal region. One side of the hook and loop fastener (i.e., either the “hook” side or the “loop” side) on a bottom surface of panels 42A and 42B is used to attach panels 42A and 42B to sections of the other side (referred to herein as the “receiving side”) of the hook and loop fastener fixedly positioned on device 20 (not shown). Alternatively, the bottom surface of panels 42A and 42B may attach to a receiving surface on a fixed object, such as the procedure table, or may include loop portions that loop through a ring located on the device 20 or on a fixed object and attach to itself via the hook and loop fastener. Similarly, panels 42C and 42D may be pulled generally downward and toward the back of the patient to retract the inguinal/thigh pannus away from the inguinal region. Hook and loop fastener on the reverse side of straps 42C and 42D is used to attach straps 42C and 42D to corresponding hook and loop fastener fixedly positioned on device 20 or a fixed object and thus maintain retraction of the inguinal/thigh pannus.

In other embodiments, other fasteners such as hook and eye fasteners, buckles, belts, clips, adhesives, or other fasteners that are known or will be known to those of skill in the art may be substituted for the hook and loop fastener described with respect to FIG. 4 without departing from the spirit and scope of the present invention.

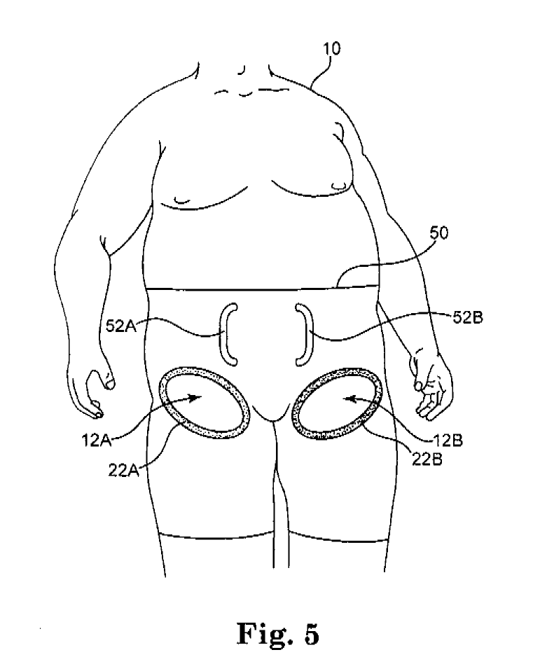

FIG. 5 is a front view of another example abdominal retraction device 50 as worn by a patient 10. In this example, device 50 includes one or more anchors 52A and 52B to which straps or other retraction mechanisms may be attached for refraction of the abdominal pannus. The one or more anchors 52A and 52B may be positioned on upper portion 31 of device 20. In the case of a single anchor, the single anchor may be positioned on upper portion 31 and aligned with the midline of device 20 and thus with the midline of patient 10.

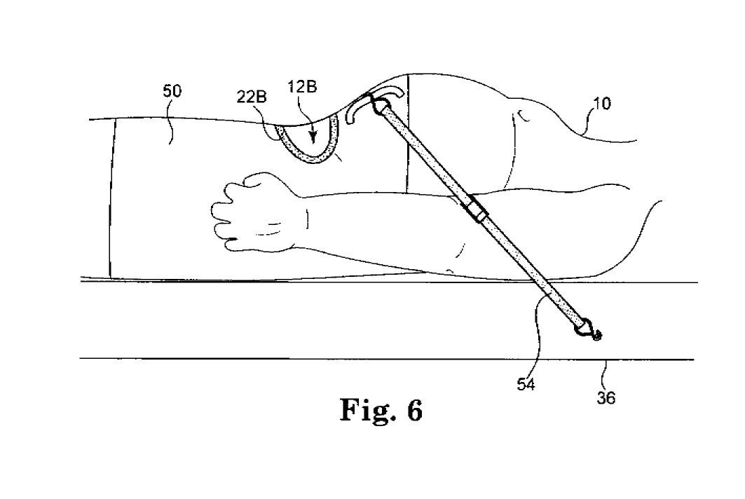

FIG. 6 is a side view of the example abdominal retraction device of FIG. 5 as worn by a patient 10 after application of additional retraction of the abdominal tissue. One or more straps 54 (similar to those described above with respect to FIG. 2B, for example) may be attached to anchor 52A (not shown) and 52B and tension applied to retract the abdominal pannus away from the relevant inguinal region(s). In this example, strap 54 retracts the abdominal pannus laterally toward the left side of the patient and also in a somewhat superior direction. In some examples, retraction to one side may be all that is required for a particular procedure. In other examples, additional straps 54 may be attached to the other anchor to retract the abdominal pannus laterally toward the left side of the patient (not shown). Some combination of multiple straps applying retraction in multiple directions may also be used. As with FIG. 2B, straps 54 may be secured to procedure table 36 using cleats, rails or other attachment mechanisms on procedure table 36.

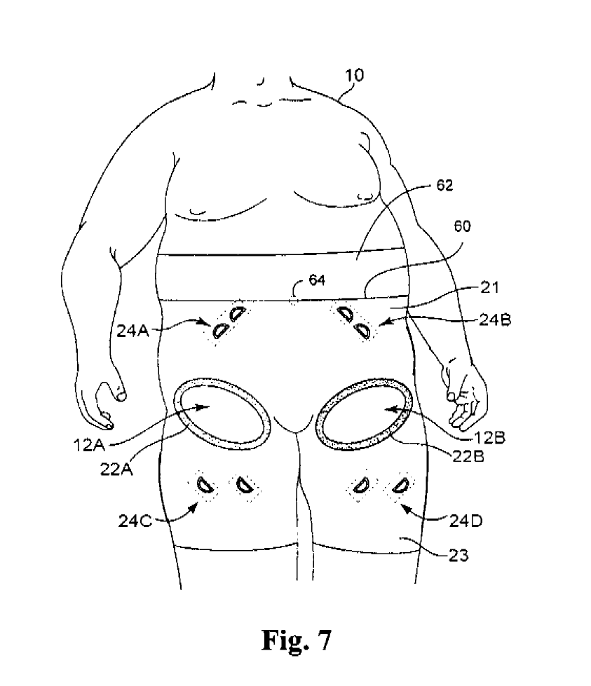

FIG. 7 is a front view of another example abdominal retraction device 60 as worn by a patient. In this example, device 60 includes a waist panel 62 that covers a greater portion of the patient's abdomen relative to what is shown as covered in FIGS. 1-6. For example, waist panel 62 may be sized to cover at least the patient's umbilicus, the position of which is indicated generally by reference number 64. In general, when worn by a patient, waist panel 62 may be sized to cover at least the umbilicus of the patient and may cover as far as up to the inferior margins of the ribs and sternum.

To accomplish this, the width of waist panel 62 (as measured from the top of the waist band of upper portion 21 to a top edge of waist panel 62) may be sized anywhere from about 2 inches to about 10 inches, or any width that is suitable to more completely cover the abdomen of a majority of patients. In other words, the “rise” of device 60, as measured by the distance between the crotch and the top edge of waist panel 62, may vary from between about 12 inches (or around umbilicus height, depending upon the size of the patient) to about 30 inches.

As shown in the example of FIG. 7, waist panel 62 may be a separate piece of fabric that is sewn or otherwise joined with the top edge (around the waist band) of upper portion 21. In another example (not shown) instead of a separate waist panel 62, the upper portion 21 may be constructed such that the top edge of upper portion 21 covers a greater portion of the patient's abdomen. In other words, waist panel 62 may be a separate panel of fabric as shown in FIG. 7, or, the waist line of upper portion 21 may simply be extended higher to cover a greater portion of the patient's abdomen.

In the example of FIG. 7, device 60 includes multiple sets of fasteners 24A-24D. Each set of fasteners 24A-24D includes two fasteners along with webbing which aids in attaching fasteners 24A-24D to device 60 and also reinforces the fabric to prevent tearing during application of retraction forces to the fasteners using belts, straps, etc. However, it shall be understood that each set of fasteners may include one or more individual fasteners and/or one or more rows of fasteners, and that the invention is not limited in this respect.

Fasteners 24A-24D may be attached to the upper portion 21 of device 60, as shown in FIG. 7, or may be attached to waist panel 62, or there may be fasteners present on both upper portion 21 and waist panel 62. Device 60 may also include variously located fastening mechanisms to which belts, tie downs or other straps may be attached, the use of bib or shoulder straps such as those shown in FIG. 3, the use of hook and loop fastener straps such as those shown in FIG. 4, or any combination of these. In the case of bib or shoulder straps, the straps may be attached to either the upper portion 21 or to the waist panel 62.

In general, the upper and lower portions 21, 23 of abdominal retraction devices such as 20, 40, 50 or 60 shown and described herein, or other example abdominal retraction devices which shall become apparent to those of skill in the art upon reading and understanding this specification, may be constructed using a fabric having sufficient stiffness to adequately contain the abdominal and/or thigh pannus for purposes of ensuring access to the inguinal region of a patient. One such nonlimiting example is a fabric containing 30% spandex/70% lycra. For those devices such as device 60 that include a separate waist panel 62, waist panel 62 may be constructed from the same fabric as upper and lower portions 21, 23. Alternatively, waist panel 62 may be constructed from a relatively less stiff fabric as compared to the fabric used to construct the upper and lower portions 21, 23, such as a fabric containing a relatively smaller percentage of spandex, such as 20% spandex/10% lycra, or 10% spandex/90% lycra. In general, the spandex content of waist panel 62 may be less than or equal to the spandex content of upper portion 21 in examples where the stiffness of waist panel 62 may be less than that of upper portion 21. However, it shall be understood that many other fabrics could also be used to construct an abdominal refraction device, and that the invention is not limited in this respect.

Various embodiments of the invention have been described. These and other embodiments are within the scope of the following claims.

1. An apparatus comprising a garment including an upper portion that defines a waist opening and having a front side and a back side, and a lower portion including a pair of legs that extend from the upper portion, the garment further having first and second apertures positioned on the front side of the garment such that the first and second apertures expose the right and left inguinal regions of a patient, respectively, when the patient wears the garment.

2. The apparatus of claim 1, further comprising a first upper fastening mechanism positioned on the right front side of the upper portion and a second upper fastening mechanism positioned on the left front side of the upper portion.

3. The apparatus of claim 2, further comprising at least one retraction strap releasably connected to one of the first or second upper fastening mechanisms.

4. The apparatus of claim 3, wherein the at least one retraction strap applies retraction tension to the upper portion of the garment via the first or second fastening mechanism, such that an abdominal pannus of the patient is refracted away from the right or left side, respectively, of the inguinal region.

5. The apparatus of claim 4, wherein the retraction strap further includes connectors that releasably attach to one of a procedure table, a fixed object in a procedure room, or a portion of the garment.

6. The apparatus of claim 2, the garment further including a first lower fastening mechanism positioned on the right front side of the lower portion and a second lower fastening mechanism positioned on the left front side of the lower portion.

7. The apparatus of claim 6, the garment further including a plurality of retraction straps that each releasably connect to one of the first upper fastening mechanism, the second upper fastening mechanism, the first lower fastening mechanism or the second lower fastening mechanism.

8. The apparatus of claim 7, wherein each of plurality of retraction straps further includes connectors that releasably attach to a fixed object in a procedure room.

9. The apparatus of claim 8, wherein the fixed object in the procedure room is a procedure table.

10. The apparatus of claim 7, wherein the retraction straps are adjustable.

11. The apparatus of claim 2, wherein the first upper fastening mechanism and the second upper fastening mechanism each comprise one or more hooks, D-rings, snaps, buckles, anchors, or buttons.

12. The apparatus of claim 2, wherein the first upper fastening mechanism and the second upper fastening mechanism each comprise a fabric panel comprising hook and loop fastener.

13. The apparatus of claim 1, the garment further including first adhesive regions on the outside surface of the garment that surround the first and second apertures, respectively.

14. The apparatus of claim 13 wherein the first adhesive regions comprise one of glue, tape or hook and loop fastener.

15. The apparatus of claim 1, the garment further including second adhesive regions on the inside surface of the garment that surrounds the first and second apertures, respectively.

16. The apparatus of claim 15, wherein the second adhesive regions comprise one of glue or tape.

17. The apparatus of claim 1, the garment further comprising an anchor positioned at the midline of the front side of the upper portion of the garment.

18. The apparatus of claim 17, further comprising at least one retraction strap releasably connected at a first end to a first side of the anchor, the at least one retraction strap including a connector at a second end that releasably attaches to a right or left side of a procedure table such that an abdominal pannus of the patient is retracted away from the right or left inguinal region, respectively.

19. The apparatus of claim 1, the garment further comprising first and second anchors positioned on the front side of the upper portion of the garment.

20. The apparatus of claim 19, further comprising a first retraction strap releasably connected at a first end to the first anchor, the first retraction strap including a connector at a second end that releasably connects to a fixed object, and comprising a second retraction strap releasably connected to the second anchor, the second retraction strap including a connector at a second end that releasably connects to a fixed object.

21. The apparatus of claim 1, the garment further comprising a waist panel extending from a top edge of the upper portion of the garment that covers at least the umbilicus of the patient.

22. The apparatus of claim 21, wherein the waist panel covers at most the inferior margins of the ribs and sternum.

23. The apparatus of claim 1, wherein the upper and lower portions of the garment comprise a fabric having a spandex content of approximately 30%.

24. The apparatus of claim 22, the garment further comprising a waist panel extending from a top edge of the upper portion of the garment that covers at least the umbilicus of the patient, the waist panel having a spandex content less than or equal to the spandex content of the upper and lower portions of the garment.

25. The apparatus of claim 1, wherein the garment is comprised of a radiolucent material.

26. The apparatus of claim 1, further comprising a plurality of fastening mechanisms to which retraction force may be applied, each of the plurality of fastening mechanisms comprised of a radiolucent material.
